(19) United States
(12) Patent Application Publication
(10) Pub. No.: US 2026/0008754 A1
(43) Pub. Date: Jan. 8, 2026

(54) N-SUBSTITUTED DERIVATIVES OF L-(L-PHENYL-3-ARYL)-LFF-PYRAZOL-4-YL)METHANAININE, METHOD FOR THEIR PRODUCTION AND THEIR APPLICATIONS

(71) Applicant: Uniwersytet Medyczny w Lublinie, Lublin (PL)

(72) Inventors: Damian Bartuzi, Lublin (PL); Tomasz M. Wrobel, Rzeszów (PL); Wojciech Tupik, Lancut (PL); Sylwia Wozniak, Niemce (PL); Agnieszka A. Kaczor, Lublin (PL); Dariusz Matosiuk, Lublin (PL)

(21) Appl. No.: 19/133,866

(22) PCT Filed: Nov. 28, 2023
(86) PCT No.: PCT/PL2023/000055

§ 371 Date: May 29, 2025

(30) Foreign Application Priority Data
Nov. 29, 2022 (PL) P.442982

(51) Int. Cl. C07D 231/12; A61K 31/415; A61K 31/4155; A61K 31/4439; A61K 31/519; C07D 401/04; C07D 401/14; C07D 405/12; C07D 405/14; C07D 487/04

(52) U.S. Cl. CPC C07D 231/12; A61K 31/415; A61K 31/4155; A61K 31/4439; A61K 31/519; C07D 401/04; C07D 401/14; C07D 405/12; C07D 405/14; C07D 487/04

(57) ABSTRACT

The subject of the invention is N-substituted derivatives of l-(l-phenyl-3-aryl)-lH-pyrazol-4-yl)methanamine with the general formula 1, where ¥ represents CH or N, R1 represents hydrogen, R2 represents no substituent, R3 represents hydrogen, while R4 represents N-propylbenzamide, benzo[b]furan, dihydrobenzo [b] furan, methylenedioxybenzene, ethyl benzoate, or propyl benzoate, substituted accordingly, their method of production and application, and the method of obtaining and application of N-substituted derivatives of l-(l-phenyl-3-aryl)-IH-pyrazol-4-yl)methanamine with the general formula 1, where Y represents C, CH, or N, R1 represents hydrogen or a methyl group, R2 represents a methyl group or no substituent, R3 represents hydrogen or a methyl group, while R4 represents dimethylpyrazole, methylpyrazole, dimethylimidazo[l,2-a]pyrimidine, or isopropoxybenzene, substituted accordingly. The compounds that are the subject of the invention are obtained through reductive amination using borohydride derivatives as the reducing agent, advantageously sodium triacetoxyborohydride, in the amount of 1.4-2.0 eq, amine in the amount of 1.0-1.1 eq, advantageously in slight excess, base, advantageously triethylamine in the amount of 1.0-1.1 eq (in the case of using amine in the form of hydrochloride), and the starting aldehyde. The reaction is conducted in a nonpolar solvent environment, advantageously dichloroethane at a concentration of about 0.1 M. The crude product is purified by column chromatography on silica gel using a methanol in dichloromethane mixture as the eluent, in concentration ranges from 2% to 5%. In order to obtain a solid form and improve the physicochemical parameters, the free bases are converted into hydrochloride form, and then crystallized from a polar solvent, advantageously ethanol or propanol.

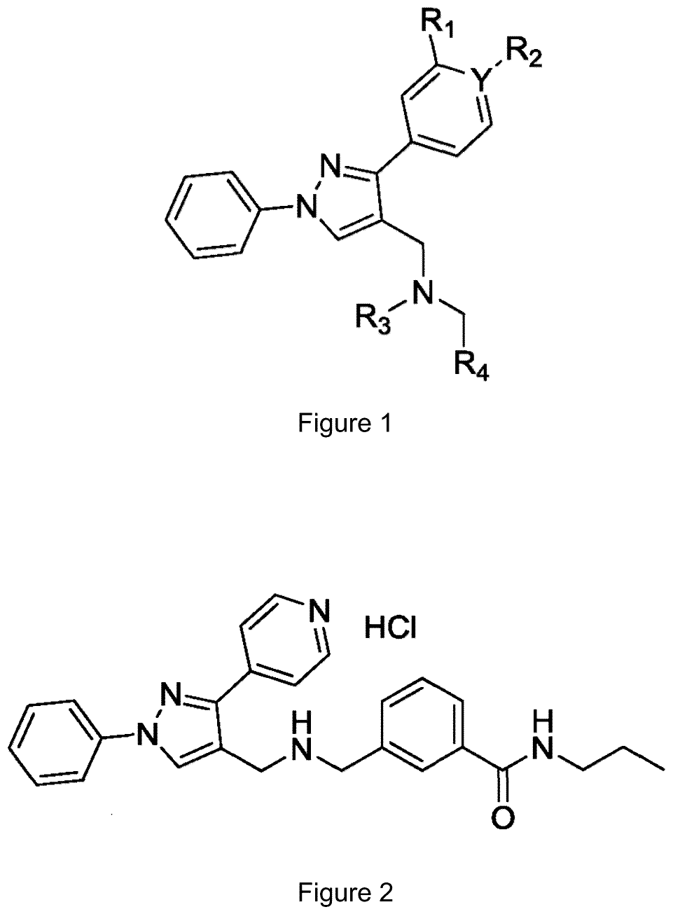

The invention relates to N-substituted derivatives of 1-(1-phenyl-3-aryl)-1H-pyrazol-4-yl) methanamine of the general formula 1, where Y represents C, CH, or N, R1 represents hydrogen or a methyl group, R2 represents a methyl group or the absence of a substituent, R3 represents hydrogen or a methyl group, while R4 represents respectively substituted N-propylbenzamide, benzo[b]furan, dihydrobenzo[b]furan, ethyl benzoate, propyl benzoate, dimethylpyrazole, methylpyrazole, dimethylimidazo[1,2-a]pyrimidine or isopropoxybenzene, and the method of their preparation and their use.

In the known state of the art, there are no reports regarding N-substituted derivatives of 1-(1-phenyl-3-aryl)-1H-pyrazol-4-yl)methanamine according to the invention, their method of preparation, and application.

The invention addresses the problem of obtaining compounds that are agonists of the u opioid receptor, some of which exhibit biased agonism, i.e., they are functionally selective towards the G-protein signaling pathway relative to β-arrestin (Table 4).

The compounds of the invention are obtained via reductive amination using borohydride derivatives as the reducing agent, preferably sodium triacetoxyborohydride in the amount of 1.4-2.0 eq, amine in the amount of 1.0-1.1 eq, preferably in slight excess, base, preferably triethylamine in the amount of 1.0-1.1 eq (in case of using amine in the form of hydrochloride), and the starting aldehyde. The reactions are carried out in the environment of a non-polar solvent, preferably dichloroethane at a concentration of about 0.1 M. The crude product is purified by column chromatography on silica gel using a mixture of methanol in dichloromethane as the eluent, preferably from 2% to 5%. In order to obtain a solid form and improve physicochemical parameters, the free bases are converted into hydrochloride form using an anhydrous solution of hydrogen chloride in ethanol (1.25 M), and then crystallized from a polar solvent, preferably ethanol or propanol.

Compounds of the general formula 1 according to the invention for use in the treatment of pain of varied etiology with simultaneous reduction of side effects such as tolerance, addiction, respiratory depression, or acute constipation, as well as in perioperative and intraoperative analgesia and anesthesia, and in the treatment of addiction to drugs and psychoactive substances acting through interaction with opioid receptors. Compounds of the general formula 1 may also be used to assist in the treatment of pain of varied etiology with simultaneous reduction of side effects such as tolerance, addiction, respiratory depression, or acute constipation, as well as in perioperative and intraoperative analgesia and anesthesia, and in assisting the treatment of addiction to drugs and psychoactive substances acting through interaction with opioid receptors.

The compounds according to the invention are agonists of the μ opioid receptor, some of which exhibit biased agonism, i.e., they are functionally selective towards the G-protein signaling pathway relative to β-arrestin (Table 4). Activation of the former is responsible for the analgesic effect, while the latter influences the development of a series of undesirable actions such as tolerance or addiction (Burat et al., 2020). The compounds of the invention may be used for treating or assisting in the treatment of pain and have an advantage over traditional drugs acting through opioid receptors, as they do not cause a series of side effects, such as tolerance, addiction, respiratory depression, or acute constipation.

### EXAMPLE OF SYNTHESIS OF COMPOUND 8

In a dry 25 mL flask, 1,3-diphenyl-1H-pyrazole-4-carbaldehyde (248 mg, 1.00 mmol) was mixed with propyl 3-(aminomethyl)benzoate hydrochloride (253 mg, 1.10 mmol, 1.1 eq), triethylamine (111 mg, 153 μL, 1.10 mmol, 1.1 eq) in dichloroethane (10 mL, 0.1 M) for 5 minutes at room temperature. Subsequently, sodium triacetoxyborohydride (424 mg, 2.00 mmol, 2 eq) was added and the mixture was stirred for an additional 14 hours at room temperature. After this time, the reaction mixture was diluted with dichloromethane (10 mL) and washed with water in a separatory funnel (20 mL). The aqueous layer was extracted using a fresh portion of dichloromethane (20 mL). The combined organic layers were dried with anhydrous magnesium sulfate. The crude product was purified by column chromatography on silica gel, eluting with a 2% methanol solution in dichloromethane (colorless liquid, 226 mg). The purified product was converted into the hydrochloride form by dissolving in ether and adding anhydrous hydrochloric acid in ethanol. The final product was recrystallized from propanol (white precipitate, 117 mg).

### EXAMPLE OF SYNTHESIS OF COMPOUND 11

In a 25 mL flask, 1-phenyl-3-(pyridin-4-yl)-1H-pyrazole-4-carbaldehyde (249 mg, 1.00 mmol), ethyl 3-(aminomethyl)benzoate hydrochloride (216 mg, 1.00 mmol, 1 eq), sodium triacetoxyborohydride (297 mg, 1.40 mmol, 1.4 eq), and triethylamine (101 mg, 139 μL, 1.00 mmol, 1 eq) were mixed in dichloroethane (10 mL, 0.1 M) for 14 hours at room temperature. After this time, the reaction mixture was diluted with dichloromethane (10 mL) and washed with a saturated sodium carbonate solution in a separatory funnel. The organic layer was acidified with 1M hydrochloric acid solution and washed twice with dichloromethane. Then, the aqueous layer was made alkaline with a 5% sodium hydroxide solution and extracted twice with dichloromethane. The combined organic layers were dried with anhydrous magnesium sulfate. The crude product was purified by column chromatography on silica gel, eluting with a 2% methanol solution in dichloromethane (yellow liquid, 246 mg). The purified product was converted into the hydrochloride form by dissolving in ether and adding anhydrous solution of hydrogen chloride in ethanol. The final product was recrystallized from ethanol (white precipitate, 190 mg).Physicochemical, spectral, and pharmacological data of the N-substituted derivatives of 1-(1-phenyl-3-aryl)-1H-pyrazol-4-yl)methanamine, obtained as described in the examples, are presented in the Tables.

TABLE 1
New compounds that are the subject of the invention, including commercially
available compounds.

| | | | | | | Commercially |
|---|---|---|---|---|---|---|
| No. | Y | R₁ | R₂ | R₃ | R₄ | Available Substance |
| 1 2 | N CH | H | — | H | 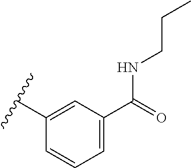 [SMILES: *c1cccc(C(=O)NCCC)c1] | Yes No |
| 3 4 | N CH | H | — | H | 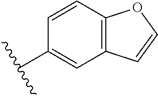 [SMILES: *c1ccc2occc2c1] | Yes No |
| 5 | C | CH₃ | CH₃ | H | H | Yes |
| 6 | CH | H | — | | 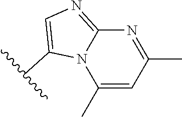 [SMILES: *c1cnc2nc(C)cc(C)n12] | Yes |
| 7 | CH | H | — | CH₃ | 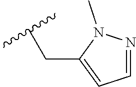 [SMILES: *Cc1ccnn1C] | Yes |
| 8 | CH | H | — | H | | No |
| 9 | N | | | | 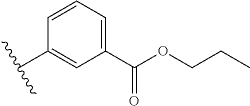 [SMILES: *c1cccc(C(=O)OCCC)c1] | No |
| 10 11 | CH N | H | — | H | 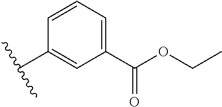 [SMILES: *c1cccc(C(=O)OCC)c1] | No No |
| 12 | N | H | — | H | 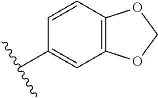 [SMILES: *c1ccc2c(c1)OCO2] | No |
| 13 | | | | | 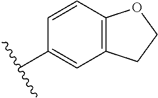 [SMILES: *c1ccc2c(c1)CCO2] | No |
| 14 | | | | | 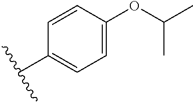 [SMILES: *c1ccc(OC(C)C)cc1] | Yes |
| 15 | | | | | 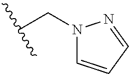 [SMILES: *Cn1cccn1] | Yes |

TABLE 2
Characteristics and details of the purification procedure for the derivatives that
are the subject of the invention.

| | | | | | | Molecular | Molecular | Yield | Purification |
|---|---|---|---|---|---|---|---|---|---|
| No. | Y | R₁ | R₂ | R₃ | R₄ | Formula | Weight | [%] | Method |
| 1 2 | N CH | H | — | H | 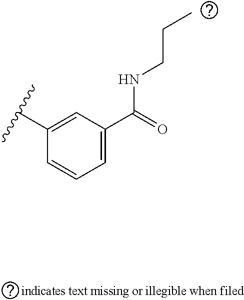 [SMILES: *c1cccc(C(=O)NCCC)c1] | C₂₆H₂₇N₅O C₂₇H₂₈N₄O | 425.536 424.548 | 32 29 | chromatography/ crystallization chromatography/ crystallization |
| 3 4 | N CH | H | — | H | 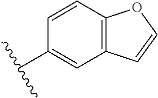 [SMILES: *c1ccc2occc2c1] | C₂₄H₂₀N₄O C₂₅H₂₁N₃O | 380.451 379.463 | 25 25 | chromatography/ crystallization chromatography/ crystallization |
| 5 | C | CH₃ | CH₃ | H | H | C₁₉H₂₁N₃ | 291.398 | — | — |
| 6 | CH | H | — | | 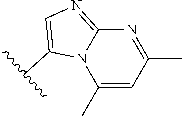 [SMILES: *c1cnc2nc(C)cc(C)n12] | C₂₅H₂₄N₆ | 408.509 | — | — |
| 7 | CH | H | — | CH₃ | 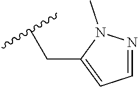 [SMILES: *Cc1ccnn1C] | C₂₃H₂₅N₅ | 371.488 | — | — |
| 8 9 | CH N | H | — | H | 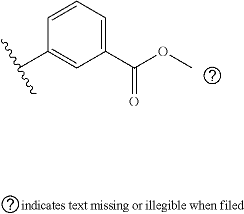 [SMILES: *c1cccc(C(=O)OC)c1] | C₂₇H₂₇N₃O₂ C₂₆H₂₆N₄O₂ | 425.532 426.52 | 53 24 | crystallization chromatography/ crystallization |
| 10 11 | CH N | H | — | H | 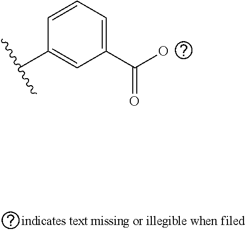 [SMILES: *c1cccc(C(=O)O)c1] | C₂₆H₂₅N₃O₂ C₂₅H₂₄N₄O₂ | 411.505 412.493 | 49 60 | chromatography/ crystallization crystallization |
| 12 | N | H | — | H | 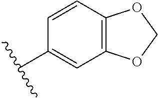 [SMILES: *c1ccc2c(c1)OCO2] | C₂₃H₂₀N₄O₂ | 384.439 | 36 | chromatography/ crystallization |
| 13 | | | | | 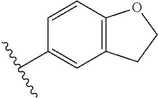 [SMILES: *c1ccc2c(c1)CCO2] | C₂₄H₂₂N₄O | 382.467 | 44 | chromatography/ crystallization |
| 14 | | | | | 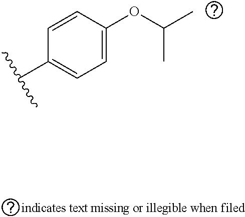 [SMILES: *c1ccc(OC(C)C)cc1] | C₂₅H₂₆N₄O | 398.51 | — | — |
| 15 | | | | | 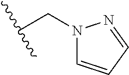 [SMILES: *Cn1cccn1] | C₂₀H₂₀N₆ | 344.422 | — | — |

TABLE 3
Spectral data for the substances that are the subject of the invention.

| No. | Spectral Data |
|---|---|
| 1 | ¹H NMR (600 MHz, MeOD) δ 8.92-8.88 (m, 2H); 8.79 (s, 1H); 8.49-8.44 (m, |
| | 2H); 8.05 (t, J = 1.6 Hz, 1H); 7.94-7.90 (m, 2H); 7.90-7.86 (m, 1H); 7.78-7.75 |
| | (m, 1H); 7.60-7.54 (m, 3H); 7.46 (tt, J = 7.1; 1.1 Hz, 1H); 4.67 (s, 2H); 4.49 (s, |
| | 2H); 3.36-3.33 (m, 2H); 1.64 (h, J = 7.4 Hz. 2H); 0.97 (t, J = 7.4 Hz, 3H). |
| | ¹³C NMR (151 MHz, MeOD) δ 169.32; 151.08; 147.17; 143.25; 140.42; 136.95; |
| | 134.29; 133.63; 132.78; 130.97; 130.62; 130.40; 129.46; 129.36; 126.25; |
| | 120.65; 115.16; 52.01; 42.91; 42.09; 23.73; 11.82. |
| 2 | ¹H NMR (600 MHz, MeOD) δ 8.65 (s, 1H); 8.00 (s, 1H); 7.87 (d, J = 7.8 Hz, 1H); |
| | 7.85-7.81 (m, 2H); 7.67-7.58 (m, 3H); 7.54-7.43 (m, 6H); 7.37 (t, J = 7.4 Hz, |
| | 1H); 4.39 (s, 2H); 4.31 (s, 2H); 3.36-3.33 (m, 2H); 1.65 (h, J = 7.4 Hz. 2H); 0.98 |
| | (t, J = 7.4 Hz, 3H). |
| | ¹³C NMR (151 MHz, MeOD) δ 169.13; 153.76; 140.80; 136.64; 134.13; 133.15; |
| | 132.71; 131.56; 130.68; 130.43; 130.25; 129.93; 129.74; 129.42; 129.20; |
| | 128.28; 120.25; 112.57; 51.51; 42.82; 42.09; 23.64; 11.81. |
| 3 | ¹H NMR (600 MHz, MeOD) δ 8.59 (s, 1H); 8.52-8.48 (m, 2H); 7.89-7.83 (m, |
| | 3H); 7.77 (d, J = 1.6 Hz, 1H); 7.64-7.62 (m, 2H); 7.59 (d, J = 8.5 Hz, 1H); 7.54 (td, |
| | J = 7.6. 1.9 Hz, 2H); 7.45-7.39 (m, 2H); 6.90 (dd, J = 2.2; 0.9 Hz, 1H); 4.45 (s, |
| | 2H); 4.42 (s, 2H). |
| | ¹³C NMR (151 MHz, MeOD) δ 156.87; 150.60; 150.25; 148.16; 142.23; 140.75; |
| | 132.14; 130.86; 129.84; 128.85; 127.41; 126.76; 124.65; 124.01; 120.43; |
| | 113.60; 113.14; 107.71; 52.19; 41.39. |
| 4 | ¹H NMR (600 MHz, MeOD) δ 8.54 (s, 1H); 7.85 (d, J = 2.2 Hz, 1H); 7.83-7.80 (m, |
| | 2H); 7.71 (d, J = 1.6 Hz, 1H); 7.57-7.50 (m, 5H); 7.42-7.35 (m, 5H); 6.87 (dd, |
| | J = 2.2; 0.9 Hz. 1H; 4.37 (s, 2H); 4.33 (s, 2H). |
| | ¹³C NMR (151 MHz, MeOD) δ 156.82; 153.94; 147.99; 140.94; 133.22; 131.47; |
| | 130.78; 129.93; 129.80; 129.77; 129.44; 128.43; 127.37; 126.63; 124.61; |
| | 120.39; 113.05; 112.68; 107.74; 51.95; 41.55. |
| 8 | ¹H NMR (600 MHz, MeOD) δ 8.57 (s, 1H); 8.13 (t, J = 1.8 Hz, 1H); 8.09 (dt, J = |
| | 7.8; 1.4 Hz, 1H); 7.85-7.81 (m, 2H); 7.71 (ddd, J = 7.7; 1.9; 1.2 Hz, 1H); 7.61- |
| | 7.53 (m, 5H); 7.49-7.44 (m, 3H); 7.43-7.39 (m, 1H); 4.40 (s, 2H); 4.33-4.29 (m, |
| | 4H); 1.81 (dtd, J = 14.0; 7.4; 6.6 Hz, 2H); 1.05 (t, J = 7.5 Hz, 3H). |
| | ¹³C NMR (151 MHz, MeOD) δ 166.17; 152.63; 139.43; 134.46; 131.67; 131.48; |
| | 131.17; 130.61; 130.21; 130.18; 129.43; 129.33; 128.64; 128.52; 128.10; |
| | 127.15; 119.10; 111.16; 66.82; 49.92; 40.53; 21.72; 9.46. |
| 9 | ¹H NMR (600 MHz, MeOD) δ 8.66 (s, 1H); 8.63-8.59 (m, 2H); 8.19 (s, 1H); 8.10- |
| | 8.06 (m, 1H); 7.89-7.84 (m, 2H); 7.78 (d, J = 7.8 Hz, 1H); 7.76-7.72 (m, 2H); |
| | 7.60-7.51 (m, 3H); 7.40 (t, J = 7.4 Hz, 1H); 4.49 (s, 2H); 4.41 (s, 2H); 4.28 (t, J = |
| | 6.6 Hz, 2H); 1.79 (h, J = 7.3 Hz, 2H); 1.03 (t, J = 7.4 Hz, 3H). |
| | ¹³C NMR (151 MHz, MeOD) δ 167.29; 150.53; 150.14; 142.39; 140.70; 135.84; |
| | 133.11; 132.67; 132.27; 132.09; 131.56; 130.82; 130.66; 128.79; 124.14; |
| | 120.40; 113.52; 68.08; 51.61; 42.04; 23.15; 10.82. |
| 10 | ¹H NMR (600 MHz, MeOD) δ 8.60 (d, J = 0.7 Hz, 1H); 8.14 (q, J = 1.3 Hz, 1H); |
| | 8.06 (dt, J = 8.1; 1.4 Hz, 1H); 7.85-7.80 (m, 2H); 7.72-7.69 (m, 1H); 7.61-7.57 |
| | (m, 2H); 7.56-7.49 (m, 3H); 7.47-7.40 (m, 3H); 7.39-7.34 (m, 1H); 4.40-4.34 |
| | (m, 4H); 4.31 (s, 2H); 1.38 (td, J = 7.1; 0.6 Hz, 3H). |
| | ¹³C NMR (151 MHz, MeOD) δ 165.84; 152.49; 139.49; 134.40; 131.82; 131.45; |
| | 131.26; 130.67; 130.20; 130.16; 129.37; 129.21; 128.59; 128.43; 128.09; |
| | 127.01; 118.97; 111.09; 61.12; 49.95; 40.69; 13.23. |
| 11 | ¹H NMR (600 MHz, MeOD) δ 8.95-8.92 (m, 2H); 8.81 (s, 1H); 8.54-8.49 (m, |
| | 2H); 8.25 (t, J = 1.8 Hz, 1H); 8.11 (dt, J = 7.9; 1.4 Hz, 1H); 7.96-7.92 (m, 2H); 7.88 |
| | (dt, J = 7.7; 1.5 Hz, 1H); 7.63-7.58 (m, 3H); 7.50-7.45 (m, 1H); 4.71 (s, 2H); 4.53 |
| | (s, 2H); 4.40 (q, J = 7.1 Hz, 2H); 1.41 (t, J = 7.1 Hz, 3H). |
| | ¹³C NMR (151 MHz, MeOD) δ 167.27; 151.07; 147.17; 143.26; 140.40; 135.93; |
| | 133.59; 133.01; 132.70; 132.18; 131.62; 130.96; 130.67; 129.45; 126.28; |
| | 120.61; 115.13; 62.56; 51.86; 42.23; 14.63. |
| 12 | ¹H NMR (600 MHz, D₂O) δ 8.82-8.78 (m, 2H); 8.47 (s, 1H); 8.15-8.10 (m, 2H); |
| | 7.80-7.75 (m, 2H); 7.64-7.58 (m, 2H); 7.55-7.50 (m, 1H); 6.87 (d, J = 1.1 Hz, |
| | 2H); 6.82 (t, J = 1.0 Hz, 1H); 6.00 (s, 2H); 4.47 (s, 2H); 4.20 (s, 2H). |
| | ¹³C NMR (151 MHz. D₂O) δ 151.18; 151.13; 150.65; 149.67; 145.20; 141.46; |
| | 135.53; 132.88; 131.67; 127.95; 127.26; 126.54; 123.19; 115.86; 112.84; |
| | 111.94; 104.68; 52.83; 41.73. |
| 13 | ¹H NMR (600 MHz, D₂O) δ 8.79-8.74 (m, 2H); 8.42 (s, 1H); 8.10-8.06 (m, 2H); |
| | 7.79-7.73 (m, 2H); 7.63-7.57 (m, 2H); 7.55-7.49 (m, 1H); 7.24-7.20 (m, 1H); |
| | 7.10 (dd, J= 8.2. 2.0 Hz, 1H); 6.77 (d, J = 8.2 Hz, 1H); 4.60 (t, J = 8.8 Hz, 2H); 4.47 |
| | (s, 2H); 4.23 (s, 2H); 3.16 (t, J = 8.8 Hz, 2H). |
| | ¹³C NMR (151 MHz. D₂O) δ 162.92; 151.29; 149.43; 144.96; 141.40; 135.48; |
| | 133.17; 132.87; 131.89; 131.63; 129.96; 127.87; 125.32; 123.04; 116.01; |
| | 112.50; 75.07; 52.86; 41.57; 31.62. |

TABLE 4
Pharmacological properties of the derivatives against the μ opioid receptor.

| | EC₅₀ | EC₅₀ | Biased Agonism | | | |
|---|---|---|---|---|---|---|
| | (G protein) | (β arrestin) | Selectivity | | | |
| No. | [nm] | [nm] | Coefficient k | Yes/No | LogP | LogBBB |
| 1 | 703.6 | 18800.0 | 26.7 | No | 4.849 | −0.368 |
| 2 | 145.5 | 5060.0 | 34.8 | No | 5.954 | −0.284 |
| 3 | 1.235 | 195.0 | 157.9 | Yes | 5.037 | 0.233 |
| 4 | 139.5 | 4570.0 | 32.8 | No | 6.015 | 0.544 |
| 5 | 30400000.0 | — | — | — | 4.501 | 0.533 |
| 6 | 2186 | — | — | — | 5.240 | −0.111 |
| 7 | 538.0 | — | — | — | 5.444 | 0.216 |
| 8 | 6448.0 | 569.0 | 11.3 | No | 5.871 | −0.396 |
| 9 | 22210.0 | 1630.0 | 13.6 | No | 4.982 | −0.537 |
| 10 | 13950.0 | 1830.0 | 7.6 | No | 5.818 | −0.289 |
| 11 | 7220.0 | 67500.0 | 9.3 | No | 4.891 | −0.499 |
| 12 | 10.5 | 23400.0 | 2228.6 | Yes | 4.216 | 0.294 |
| 13 | 6.26 | 5890.0 | 940.9 | Yes | 4.878 | 0.211 |
| 14 | 521.0 | — | — | — | 5.546 | −0.043 |
| 15 | 3096.0 | — | — | — | 4.071 | 0.051 |

The log BBB parameter denotes the calculated permeability of the compound through the blood-brain barrier. The log P parameter denotes the calculated lipophilicity.

1. N-substituted derivatives of 1-(1-phenyl-3-aryl)-1H-pyrazol-4-yl)methanamine with the general formula 1, where Y represents CH or N, R1 represents hydrogen, R2 represents no substituent, R3 represents hydrogen, while R4 represents N-propylbenzamide, benzo[b]furan, methylenedioxybenzene, dihydrobenzo[b]furan, ethyl benzoate or propyl benzoate, respectively, substituted accordingly.

2. A method for obtaining N-substituted derivatives of 1-(1-phenyl-3-aryl)-1H-pyrazol-4-yl)methanamine with the general formula 1, where Y represents C, CH or N, R1 represents hydrogen or a methyl group, R2 represents a methyl group or no substituent, R3 represents hydrogen or a methyl group, while R4 represents N-propylbenzamide, benzo[b]furan, dihydrobenzo[b]furan, methylenedioxybenzene, ethyl benzoate, propyl benzoate, dimethylpyrazole, methylpyrazole, dimethylimidazo[1,2-a]pyrimidine or isopropoxybenzene, substituted accordingly, characterized in that 1-(1-phenyl-3-aryl)-1H-pyrazol-4-yl)methanamine with the general formula 2 is subjected to reductive amination using an amine with the appropriate aldehyde in a ratio of 1.0-1.1:1.0.

3. A method according to claim 2, characterized in that sodium triacetoxyborohydride is used as the reducing agent, in an amount of 1.4-2.0 eq.

4. A method according to claim 2, characterized in that when an amine in the form of hydrochloride is used, triethylamine is added to the reaction mixture in the amount of 1.1 eq.

5. A method according to claim 2, characterized in that the reaction is carried out in dichloroethane at room temperature.

6. A method according to claim 2, characterized in that the compounds are purified in the process of column chromatography by eluting with a mixture of methanol in dichloromethane (2-5%) on silica gel, and the final compound after conversion to hydrochloride is crystallized from ethanol or propanol.

7. N-substituted derivatives of 1-(1-phenyl-3-aryl)-1H-pyrazol-4-yl)methanamine with the general formula 1, where Y represents C, CH or N, R1 represents hydrogen or a methyl group, R2 represents a methyl group or no substituent, R3 represents hydrogen or a methyl group, while R4 represents N-propylbenzamide, benzo[b]furan, dihydrobenzo[b]furan, methylenedioxybenzene, ethyl benzoate, propyl benzoate, dimethylpyrazole, methylpyrazole, dimethylimidazo[1,2-a]pyrimidine or isopropoxybenzene, substituted accordingly, being biased agonists of the μ opioid receptor (functionally selective with respect to G protein signaling pathway relative to β-arrestin) for use in the treatment of pain (of various etiologies) with simultaneous limitation of side effects such as tolerance, dependence, respiratory depression or acute constipation, as well as in perioperative and intraoperative analgesia and anesthesia, and in the treatment of addictions to drugs and psychoactive substances acting through interaction with opioid receptors.
